(19) United States
(12) Patent Application Publication
(10) Pub. No.: US 2025/0059592 A1
(43) Pub. Date: Feb. 20, 2025

(54) SUPPRESSION OF NON-SPECIFIC SIGNALS BY EXONUCLEASES IN FISH EXPERIMENT

(71) Applicant: CALIFORNIA INSTITUTE OF TECHNOLOGY, Pasadena, CA (US)

(72) Inventors: Long CAI, Pasadena, CA (US); Chee Huat (Linus) ENG, Pasadena, CA (US)

(21) Appl. No.: 18/723,184

(22) PCT Filed: Dec. 23, 2022
(86) PCT No.: PCT/US2022/053995

§ 371 Date: Jun. 21, 2024

Related U.S. Application Data
(60) Provisional application No. 63/293,352, filed on Dec. 23, 2021.

(51) Int. Cl. C12Q 1/682

(52) U.S. Cl. CPC C12Q 1/682; C12Q 2600/166

(57) ABSTRACT

Disclosed herein is a composition for nuclease treatment of hybridized probes in experiments to profile and analyze biological samples. Also disclosed herein, is a kit for nuclease treatment of hybridized probes in experiments to profile and analyze biological samples. Also disclosed herein, is a method for nuclease treatment of hybridized probes in experiments to profile and analyze biological samples.

### CROSS-REFERENCE TO RELATED APPLICATIONS

This application claims the benefit of U.S. Provisional Patent Application No. 63/293,352, filed Dec. 23, 2021. The contents of the above-referenced application are hereby incorporated by reference in their entirety.

### STATEMENT REGARDING FEDERALLY SPONSORED RESEARCH OR DEVELOPMENT

This invention was made with government support under Grant No. HL145609 awarded by the National Institutes of Health. The government has certain rights in the invention.

### BACKGROUND

Transcription profiling of cells is valuable for many purposes. Microscopic imaging resolving multiple mRNAs in single cells can provide information regarding transcript abundance and localization, which are important for understanding the molecular basis of cell identify and developing treatment for diseases. Molecular profiling such as transcriptomic profiling of biological samples is valuable for various purposes. For example, it could allow one to assess gene expression levels to detect and identify abnormal growth states such as cancers.

Techniques such as qPCR and microarrays have been useful, but they do not reach single molecule sensitivity. Next generation sequencing, on the other hand, involves amplification of a sample and reverse transcription of mRNA that can introduce biases and inaccuracies in quantification. Moreover, sample preparation and sequencing can be time-consuming and costly. Despite the fact that imaging has been used for mRNA transcripts quantification, it is limited to a few hundred genes. Many scientific questions become accessible if thousands of genes or even the whole transcriptome can be quantified.

What is needed are better methods and systems for carrying out imaging based transcriptomic profiling at single molecule sensitivity with high accuracy in a time efficient manner.

### SUMMARY

The present disclosure provides methods, compositions, kits, and for nuclease treatment of hybridized probes in experiments to profile and analyze biological samples. This disclosure sets forth the compositions and kits, in addition to making and using the same, and other solutions to problems in the relevant field.

In some embodiments, there is provided a composition to reduce the background signal of probes hybridized to targets, wherein the composition comprises: one or more primary probes capable of binding one or more targets, wherein each primary probe comprises one or more secondary probe binding sites and/or one or more readout probe binding sites. In certain embodiments, the composition comprises optionally, one or more secondary probes, each capable of binding the primary probe or one or more targets, wherein each secondary probe comprises one or more tertiary probe binding sites or one or more readout probe binding sites. In certain embodiments, the composition comprises optionally, one or more tertiary probes, each capable of binding to the secondary probe or one or more targets, wherein each tertiary probe comprises one or more quaternary probe binding sites or one or more readout probe binding sites. In certain embodiments, the composition comprises optionally, one or more quaternary probes, each capable of binding to the tertiary probe or one or more targets, wherein each quaternary probe comprises one or more readout probe binding sites. In certain embodiments, the composition comprises, one or more readout probes capable of binding to a readout probe binding site on the one or more primary, secondary, tertiary, or quaternary probes and capable of being detected. In certain embodiments, the composition comprises one or more exonucleases each capable of removing one or more probes that bind to targets, primary, secondary, tertiary, or quaternary probes.

In some embodiments, there is provided a kit to reduce the background signal of probes hybridized to targets, comprising a plurality of probes, so that the kit comprises at least: one or more primary probes capable of binding one or more targets, wherein each primary probe comprises one or more secondary probe binding sites and/or one or more readout probe binding sites. In certain embodiments, the kit comprises optionally, one or more secondary probes, each capable of binding the primary probe or one or more targets, wherein each secondary probe comprises one or more tertiary probe binding sites or one or more readout probe binding sites. In certain embodiments, the kit comprises optionally, one or more tertiary probes, each capable of binding to the secondary probe or one or more targets, wherein each tertiary probe comprises one or more quaternary probe binding sites or one or more readout probe binding sites. In certain embodiments, the kit comprises optionally, one or more quaternary probes, each capable of binding to the tertiary probe or one or more targets, wherein each quaternary probe comprises one or more readout probe binding sites. In certain embodiments, the kit comprises one or more readout probes capable of binding to a readout probe binding site on the primary, secondary, tertiary, or quaternary probes and are capable of being detected. In certain embodiments, the kit comprises optionally, one or more exonucleases each capable of removing one or more probes that bind to targets, primary, secondary, tertiary, or quaternary probes.

In some embodiments, there is provided a method to reduce the background signal of probes hybridized to targets, comprising steps of: contacting a sample with one or more primary probes that bind one or more targets, wherein each primary probe hybridizes to a target, wherein each primary probe comprises one or more secondary probe binding sites and/or one or more readout probe binding sites. In certain embodiments, the method comprises optionally, hybridizing one or more secondary probes to the primary probes or to one or more targets; wherein each secondary probe comprises one or more tertiary probe binding sites or one or more readout probe binding sites. In certain embodiments, the method comprises optionally, hybridizing one or more tertiary probes to at least one secondary probe or one or more targets, wherein each tertiary probe comprises one or more quaternary probe binding sites or one or more readout probe binding sites. In certain embodiments, the method comprises optionally, hybridizing one or more quaternary probes to at least one tertiary probe or one or more targets, wherein each quaternary probe comprises one or more readout probe binding sites. In certain embodiments, the method comprises hybridizing readout probes capable of detection to the one or more readout probe binding sites. In certain embodiments, the method comprises contacting the sample one or more exonucleases each capable of removing probes that bind to targets, primary, secondary, tertiary, or quaternary probes after any steps (i)-(v) to reduce the background signal of probes bound to targets. In certain embodiments, the method comprises repeating any of the previous embodiments, either individually or in any combination thereof.

In some embodiments, the methods further comprise imaging the cell after contacting the sample with one or more exonuclease so that the interaction of the primary probe to the nucleic acids is detected. In certain embodiments, the method further comprises optionally repeating the contacting and imaging steps, each time with a new plurality of detectably labeled readout probes, so that a target nucleic acid in the sample/cell is described by a barcode, determined by the repeated contacting and imaging steps, that can be differentiated from the barcodes of the other target nucleic acids in the cell.

In some embodiments, the methods are used to generate probes for use in an efficient and scalable signal amplification method that can be applied to multiplexed imaging. In certain embodiments, the methods are used to generate probes for use in Fluorescence In Situ Hybridization (seqFISH). In certain embodiments, the methods are used to generate probes for use in RNA and DNA sequential Fluorescence In Situ Hybridization (seqFISH). In certain embodiments, the methods are used to generate probes for use in immunofluorescence studies.

In contrast to other methods such as hybridization chain reaction (HCR), which only allows amplification of several amplicons at once, which is extremely time consuming when imaging tens or hundreds of species from one sample, the methods disclosed herein regarding the primary, secondary, tertiary, quaternary, and readout probes are significantly simpler and less expensive to synthesize than other chemical modifications and are easily compatible with existing enzymatic probe synthesis protocols. In contrast to other methods, such as HCR, the compositions, kit, and methods presented herein do not require enzymatic activity.

### BRIEF DESCRIPTIONS OF THE DRAWINGS

FIG. 1. Different probe design used in primary probes or amplifiers in multiplexed FISH experiments. (a). Conventional probes design (b). Padlock probes design.

FIG. 2. Conventional probes or amplifiers used in branched DNA are prone to non-specific binding and increases background staining. (A), Examples of fluorescent readout probes can still contribute to background staining when the primary probes or amplifiers non-specifically bound to incorrect sequence partially or to other cellular components such as proteins and lipids. (B), Padlock probes design is used as primary probes and amplifiers together with the usage of 5′ and 3′ exonucleases to remove non-specifically bound molecules, while correctly bound probes and amplifiers are protected from exonuclease digestion.

FIG. 3. Example of workflow of hybridization based amplification with non-specific binding suppression by exonucleases. After primary probes hybridization to the target sequences, in which the target sequences can be a DNA, or RNA, or oligonucleotide conjugated to antibody, the non-specific bound primary probes are removed via exonuclease treatment. Next, the first round of amplifiers are hybridized to the 2 or more binding sites on the primary probes. The non-specific bound amplifiers are then removed via exonuclease treatment. Then the second round of amplifiers are hybridized to the first round amplifiers and again the non-specific bound amplifiers are removed via exonuclease digestion. The first and second amplifiers can be repeated for as many times as desired to increase the signal gain before flowing in the fluorescent-conjugated readout probes. DNA T4 Ligase can be easily mix in exonuclease treatment buffer to perform ligation and exonuclease digestion at the same time.

FIG. 4. 6 round amplification with exonucleases improves the signal-to-noise ratio of the sample. Left: unamplified, direct fluorescent readout from the Eef2 primary probes gives low signal-to-noise ratio. Right: Amplification by repeating the first and second round of amplifiers hybridization 3 times (total 6 rounds) together with exonucleases to remove non-specific bound amplifiers, increases the signal-to-noise ratio dramatically and improves the fluorescent dots detection of the gene Eef2.

FIG. 5. Exonuclease treatment removes the “bad” non-specific bound amplifiers.Left: No primary probes are flowed in but the “bad” amplifiers bind non-specifically, contributing to high background, rendering identifying true positive signals impossible. Mid: an addition of exonuclease treatment after the amplifiers hybridization removes these non-specific fluorescent signals. Right: with primary probes hybridized, amplified, and exonuclease treatment, true positive dots of Eef2 are now detectable.

FIG. 6. Exonuclease treatment does not affect the dots detection of genes. Perfectly bound padlock probes are protected against the exonuclease digestion (right) and give similar fluorescent dots density as the sample without any exonuclease treatment in RNA.

FIG. 7. DNA-specific exonuclease digests imperfectly bound primary padlock probes and reduces non-specific stainings. Left: Imperfect primary probes designed with 15 nt (left) to bind correctly to target gene Eef2 and 15 nt (right) random sequences caused non-specific binding to the nucleus and cytoplasm. Right: Exonuclease treatment removes the non specific stainings of those imperfect primary probes.

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### Definitions

Unless otherwise noted, terms are to be understood according to conventional usage by those of ordinary skill in the relevant art.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein, the term “LANTERN” is an acronym that refers to linked amplification of targets with hybridization.

The term “oligonucleotide” refers to a polymer or oligomer of nucleotide monomers, containing any combination of nucleobases, modified nucleobases, sugars, modified sugars, phosphate bridges, or modified bridges. Oligonucleotides can be of various lengths. In particular embodiments, oligonucleotides can range from about 2 to about 1000 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length.

As used herein, the term “probe” or “probes” refers to any molecules, synthetic or naturally occurring, that can attach themselves directly or indirectly to a molecular target (e.g., an mRNA sample, DNA molecules, protein molecules, RNA and DNA isoform molecules, single nucleotide polymorphism molecules, and etc.). For example, a probe can include a nucleic acid molecule, an oligonucleotide, a protein (e.g., an antibody or an antigen binding sequence), or combinations thereof. For example, a protein probe may be connected with one or more nucleic acid molecules to for a probe that is a chimera. As disclosed herein, in some embodiments, a probe itself can produce a detectable signal. In some embodiments, a probe is connected, directly or indirectly via an intermediate molecule, with a signal moiety (e.g., a dye or fluorophore) that can produce a detectable signal.

As used herein, the term “binding sites” refer to a portion of a probe where other molecules may bind to the probe. In certain embodiments, the binding sites of a probe bind to another molecule through a non-covalent interaction.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the term “sample” refers to a nucleic acid such as DNA, RNA, transcripts, or chromosomes. In some embodiments, the term “sample” refers to nucleic acid that has been extracted from the cell.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

As disclosed herein, the term “label” generally refers to a molecule that can recognize and bind to specific target sites within a molecular target in a cell. For example, a label can comprise an oligonucleotide that can bind to a molecular target in a cell. The oligonucleotide can be linked to a moiety that has affinity for the molecular target. The oligonucleotide can be linked to a first moiety that is capable of covalently linking to the molecular target. In certain embodiments, the molecular target comprises a second moiety capable of forming the covalent linkage with the label. In particular embodiments, a label comprises a nucleic acid sequence that is capable of providing identification of the cell which comprises or comprised the molecular target. In certain embodiments, a plurality of cells is labelled, wherein each cell of the plurality has a unique label relative to the other labelled cells.

As disclosed herein, the term “barcode” generally refers to a nucleotide sequence of a label produced by methods described herein. The barcode sequence typically is of a sufficient length and uniqueness to identify a single cell that comprises a molecular target.

As disclosed herein, the term “linked” refers to a covalent bond or non-covalent interaction between two molecules. In particular, a type of non-covalent interaction is a hybridization.

As disclosed herein, the term “cis-ligated” generally refers to the ligation of an oligonucleotide 5′ to 3′ on the same oligonucleotide.

As disclosed herein, the term “trans-ligated” generally refers to the ligation of an oligonucleotide 5′ to 3′ to a different oligonucleotide.

As disclosed herein, the term “splint probe” or “splint” refers to probes that are complementary to other probes that may hybridize or bind to the complementary probes, the other probes are not covalently linked 5′ to 3′ to each other. In certain embodiments, the term “splint probe” uses the definition and techniques of Lohman et al. Efficient DNA ligation in DNA-RNA hybrid helices by Chlorella virus DNA ligase. Nucleic Acid Research, 2014, vol. 42 No. 3 1831-1844, incorporated by its entirety.

As disclosed herein, the term “padlock probe” refers to probes that are long oligonucleotides, whose ends are complementary to adjacent target sequences. Upon hybridization to the target, the two ends are brought into contact, allowing circularization by ligation.

### EMBODIMENTS

The disclosure herein sets forth embodiments for a composition for linked amplification of targets with hybridization, comprising a plurality of probes.

The disclosure herein sets forth embodiments for a kit for linked amplification of targets with hybridization, comprising a plurality of probes.

The disclosure herein sets forth embodiments for a method for linked amplification of targets with hybridization, comprising a plurality of probes.

The disclosure herein sets forth a method to remove nonspecific binding and amplify fluorescent signals in multiplexed FISH assay with the combination usage of padlock primary probes, padlock amplifiers, and DNA-specific exonucleases.

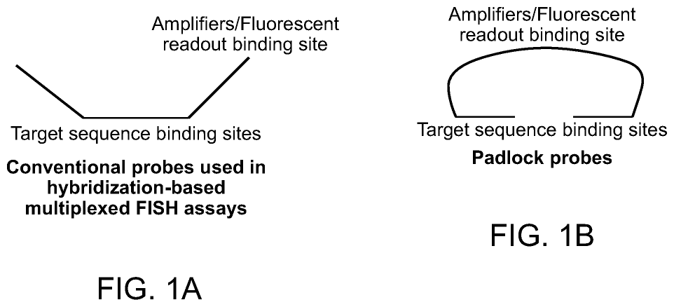

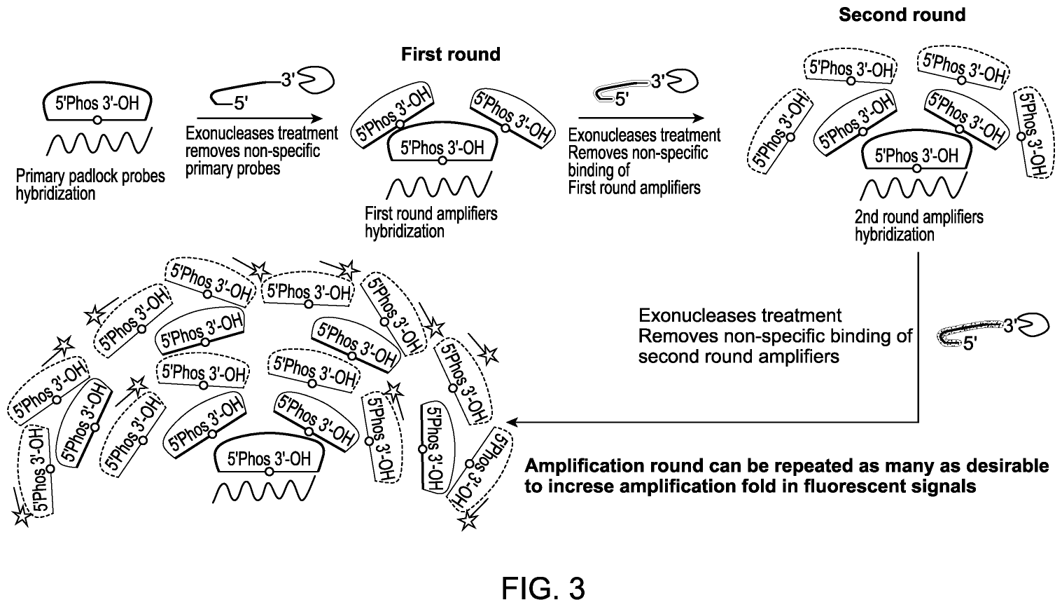

The disclosure herein sets forth an efficient and scalable signal amplification method that can be applied to multiplexed imaging such as RNA and DNA seqFISH as well as immunofluorescence (FIG. 1, FIGS. 3-5). In some embodiments, amplification of tens or hundreds of orthogonal amplicons can be performed at once.

In contrast to other methods such as hybridization chain reaction (HCR), which only allows amplification of several amplicons at once, which is extremely time consuming when imaging tens or hundreds of species from one sample, the primary, secondary, tertiary, quaternary, and readout probes are significantly simpler and less expensive to synthesize than other chemical modifications and are easily compatible with existing enzymatic probe synthesis protocols.

In some embodiments, there is provided a composition for linked-amplifying fluorescence in situ hybridization, comprising a plurality of probes, wherein the composition comprises: one or more primary probes capable of binding one or more targets, wherein each primary probe comprises one or more secondary probe binding sites and optionally one or more readout probe binding sites. In certain embodiments, the composition comprises one or more secondary probes, each capable of binding the primary probe, wherein each secondary probe comprises one or more tertiary probe binding sites or one or more readout probe binding sites. In certain embodiments, the composition optionally comprises one or more tertiary probes, each capable of binding to the secondary probe, wherein each tertiary probe comprises one or more quaternary probe binding sites or one or more readout probe binding sites. In certain embodiments, the composition optionally comprises one or more quaternary probes, each capable of binding to the tertiary probe, wherein each quaternary probe comprises one or more readout probe binding sites. In certain embodiments, the composition comprises one or more readout probes capable of binding to a binding site on the one or more primary, secondary, tertiary, or quaternary probes and capable of being detected.

In some embodiments, there is provided a kit for linked amplification of targets with hybridization, comprising a plurality of probes, wherein the composition comprises: wherein each primary probe comprises one or more secondary probe binding sites and optionally one or more readout probe binding sites. In certain embodiments, the composition comprises one or more secondary probes, each capable of binding the primary probe, wherein each secondary probe comprises one or more tertiary probe binding sites or one or more readout probe binding sites. In certain embodiments, the composition optionally comprises one or more tertiary probes, each capable of binding to the secondary probe, wherein each tertiary probe comprises one or more quaternary probe binding sites or one or more readout probe binding sites. In certain embodiments, the composition optionally comprises one or more quaternary probes, each capable of binding to the tertiary probe, wherein each quaternary probe comprises one or more readout probe binding sites. In certain embodiments, the composition comprises one or more readout probes capable of binding to a binding site on the one or more primary, secondary, tertiary, quaternary probes and capable of being detected.

In some embodiments, there is provided a method for linked amplification of targets with hybridization, comprising a method for linked amplification of targets with hybridization, comprising steps of: contacting a sample with one or more primary probes that bind one or more targets, wherein each primary probe hybridizes to the target. In certain embodiments, the method comprises hybridizing one or more secondary probes to the primary probes; wherein each secondary probe comprises one or more tertiary probe binding sites or one or more readout probe binding sites. In certain embodiments, the method optionally comprises hybridizing one or more tertiary probes to at least one secondary probe; and wherein each tertiary probe comprises one or more quaternary probes or one or more readout probe binding sites. In some embodiments, the method optionally comprises hybridizing one or more quaternary probe to at least one tertiary probe, wherein each quaternary probe comprises one or more readout probe binding sites. In certain embodiments, the method comprises hybridizing readout probes capable of detection to the one or more readout probe binding sites. In certain embodiments, the method comprises imaging the cell so that the interaction of the primary probe to the nucleic acids is detected. In certain embodiments, the method comprises optionally repeating the contacting and imaging steps, each time with a new plurality of detectably labeled readout probes, wherein at least one readout probe for one target differs from at least one other readout probes for the same target in their detectable moieties. In certain embodiments, the method of any of the previous embodiments, are repeated either individually or in any combination thereof.

In some embodiments, the methods of any of the previous embodiments comprise targeting multiple targets by probes. In some embodiments, the methods of any of the previous embodiments comprise measuring the proximity, interactions, or any combinations thereof between targets by the probes. In some embodiments, the methods of any of the previous embodiments, comprise exonuclease treatments to remove any signal that is not coming from interacting targets. In some embodiments, the probes are ligated to prevent their degradation by an exonuclease. In certain embodiments, the ligated probes are detected by rolling circle amplification (RCA) or linked amplification tethered with exponential radiance (LANTERN) to enhance detection.

In some embodiments, the methods of any of the previous embodiments, comprise ligating probes in close proximity before step (v). In certain embodiments, the methods of any of the previous embodiments comprise treating the ligated probes with an exonuclease. In certain embodiments, the methods of any of the previous embodiments comprise enhancing the signals of probes that are not cleaved after step by an exonuclease by LANTERN, RCA, branched DNA assay, HCR, signal amplification by exchange reaction (SABER), spatially-resolved transcript amplicon readout mapping (STARMAP), or any combination thereof. In certain embodiments, the methods of any of the previous embodiments comprise enhancing the signals of probes that are not cleaved after step by an exonuclease by LANTERN, RCA, branched DNA assay, HCR, SABER, STARMAP, or other in situ amplification methods. In certain embodiments, the methods of any of the previous embodiments comprise determining the proximity of the probes interacting with their targets to each other. In certain embodiments, the proximity interaction of the probes comprises glycans, cellular components, protein-protein interactions, RNA-protein interactions, RNA-RNA, RNA-DNA, DNA-protein, or combinations thereof.

### Targets of the Probes

In some embodiments, the targets are selected from transcripts, RNA, DNA loci, chromosomes, DNA, proteins, lipids, glycans, cellular target, organelles, and any combinations thereof. In certain embodiments, the transcripts, RNA, DNA loci, chromosomes, DNA, proteins, lipids, glycans, cellular target, organelles, and any combinations thereof are conjugated to an oligonucleotide. In certain embodiments, the transcripts, RNA, DNA loci, chromosomes, DNA, proteins, lipids, glycans, cellular target, organelles, and any combinations thereof are conjugated to one or more oligonucleotide sequences.

### Probes

In some embodiments, the primary probe of any of the preceding embodiments comprises oligonucleotides that are at least 10 nucleotides in length. In some embodiments, the primary probe of any of the preceding embodiments comprises oligonucleotides that are at least 11 nucleotides in length. In some embodiments, the primary probe of any of the preceding embodiments comprises oligonucleotides that are at least 12 nucleotides in length. In some embodiments, the primary probe of any of the preceding embodiments comprises oligonucleotides that are at least 13 nucleotides in length. In some embodiments, the primary probe of any of the preceding embodiments comprises oligonucleotides that are at least 14 nucleotides in length. In some embodiments, the primary probe of any of the preceding embodiments comprises oligonucleotides that are at least 15 nucleotides in length. In some embodiments, the primary probe of any of the preceding embodiments comprises oligonucleotides that are at least 16 nucleotides in length. In some embodiments, the primary probe of any of the preceding embodiments comprises oligonucleotides that are at least 17 nucleotides in length. In some embodiments, the primary probe of any of the preceding embodiments comprises oligonucleotides that are at least 18 nucleotides in length. In some embodiments, the primary probe of any of the preceding embodiments comprises oligonucleotides that are at least 19 nucleotides in length. In some embodiments, the primary probe of any of the preceding embodiments comprises oligonucleotides that are at least 20 nucleotides in length. In some embodiments, the primary probe of any of the preceding embodiments comprises oligonucleotides that are at least 21 nucleotides in length. In some embodiments, the primary probe of any of the preceding embodiments comprises oligonucleotides that are less than 30, 50, 100, 200, 250, 500, 750, or 1000 nucleotides in length.

In some embodiments, the primary probe of any of the previous embodiments comprises a nucleic acid sequence complementary to a target nucleic acid sequence.

In some embodiments, the sequence complementary to the target nucleic acid sequence is at least 60%, 65%, 70%, 75%, 80%, 85%, 90%, 91%, 92%, 93%, 94%, 95%, 96%, 97%, 98%, 99%, or 100%. In some embodiments, the sequence complementarity is at least 60%, 65%, 70%, 75%, 80%, 85%, 90%, 91%, 92%, 93%, 94%, 95%, 96%, 97%, 98%, 99%, or 100%.

In some embodiments, the secondary probe of any of the preceding embodiments comprises oligonucleotides that are at least 10 nucleotides in length. In some embodiments, the secondary probe of any of the preceding embodiments comprises oligonucleotides that are at least 11 nucleotides in length. In some embodiments, the secondary probe of any of the preceding embodiments comprises oligonucleotides that are at least 12 nucleotides in length. In some embodiments, the secondary probe of any of the preceding embodiments comprises oligonucleotides that are at least 13 nucleotides in length. In some embodiments, the secondary probe of any of the preceding embodiments comprises oligonucleotides that are at least 14 nucleotides in length. In some embodiments, the secondary probe of any of the preceding embodiments comprises oligonucleotides that are at least 15 nucleotides in length. In some embodiments, the secondary probe of any of the preceding embodiments comprises oligonucleotides that are at least 16 nucleotides in length. In some embodiments, the secondary probe of any of the preceding embodiments comprises oligonucleotides that are at least 17 nucleotides in length. In some embodiments, the secondary probe of any of the preceding embodiments comprises oligonucleotides that are at least 18 nucleotides in length. In some embodiments, the secondary probe of any of the preceding embodiments comprises oligonucleotides that are at least 19 nucleotides in length. In some embodiments, the secondary probe of any of the preceding embodiments comprises oligonucleotides that are at least 20 nucleotides in length. In some embodiments, the secondary probe of any of the preceding embodiments comprises oligonucleotides that are at least 21 nucleotides in length. In some embodiments, the secondary probe of any of the preceding embodiments comprises oligonucleotides that are less than 30, 50, 100, 200, 250, 500, 750, or 1000 nucleotides in length.

In some embodiments, the tertiary probe of any of the preceding embodiments comprises oligonucleotides that are at least 5 nucleotides in length. In some embodiments, the tertiary probe of any of the preceding embodiments comprises oligonucleotides that are at least 10 nucleotides in length In some embodiments, the tertiary probe of any of the preceding embodiments comprises oligonucleotides that are at least 11 nucleotides in length. In some embodiments, the tertiary probe of any of the preceding embodiments comprises oligonucleotides that are at least 12 nucleotides in length. In some embodiments, the tertiary probe of any of the preceding embodiments comprises oligonucleotides that are at least 13 nucleotides in length. In some embodiments, the tertiary probe of any of the preceding embodiments comprises oligonucleotides that are at least 14 nucleotides in length. In some embodiments, the tertiary probe of any of the preceding embodiments comprises oligonucleotides that are at least 15 nucleotides in length. In some embodiments, the tertiary probe of any of the preceding embodiments comprises oligonucleotides that are at least 16 nucleotides in length. In some embodiments, the tertiary probe of any of the preceding embodiments comprises oligonucleotides that are at least 17 nucleotides in length. In some embodiments, the tertiary probe of any of the preceding embodiments comprises oligonucleotides that are at least 18 nucleotides in length. In some embodiments, the tertiary probe of any of the preceding embodiments comprises oligonucleotides that are at least 19 nucleotides in length. In some embodiments, the tertiary probe of any of the preceding embodiments comprises oligonucleotides that are at least 20 nucleotides in length. In some embodiments, the tertiary probe of any of the preceding embodiments comprises oligonucleotides that are at least 21 nucleotides in length. In some embodiments, the tertiary probe of any of the preceding embodiments comprises oligonucleotides that are less than 30, 50, 100, 200, 250, 500, 750, or 1000 nucleotides in length.

In some embodiments, the quaternary probe of any of the preceding embodiments comprises oligonucleotides that are at least 5 nucleotides in length. In some embodiments, the quaternary probe of any of the preceding embodiments comprises oligonucleotides that are at least 10 nucleotides in length In some embodiments, the quaternary probe of any of the preceding embodiments comprises oligonucleotides that are at least 11 nucleotides in length. In some embodiments, the quaternary probe of any of the preceding embodiments comprises oligonucleotides that are at least 12 nucleotides in length. In some embodiments, the quaternary probe of any of the preceding embodiments comprises oligonucleotides that are at least 13 nucleotides in length. In some embodiments, the quaternary probe of any of the preceding embodiments comprises oligonucleotides that are at least 14 nucleotides in length. In some embodiments, the quaternary probe of any of the preceding embodiments comprises oligonucleotides that are at least 15 nucleotides in length. In some embodiments, the quaternary probe of any of the preceding embodiments comprises oligonucleotides that are at least 16 nucleotides in length. In some embodiments, the quaternary probe of any of the preceding embodiments comprises oligonucleotides that are at least 17 nucleotides in length. In some embodiments, the quaternary probe of any of the preceding embodiments comprises oligonucleotides that are at least 18 nucleotides in length. In some embodiments, the quaternary probe of any of the preceding embodiments comprises oligonucleotides that are at least 19 nucleotides in length. In some embodiments, the quaternary probe of any of the preceding embodiments comprises oligonucleotides that are at least 20 nucleotides in length. In some embodiments, the quaternary probe of any of the preceding embodiments comprises oligonucleotides that are at least 21 nucleotides in length. In some embodiments, the quaternary probe of any of the preceding embodiments comprises oligonucleotides that are less than 30, 50, 100, 200, 250, 500, 750, or 1000 nucleotides in length.

In some embodiments, the primary, secondary, tertiary, or quaternary probe comprises at least one readout probe binding site. In certain embodiments, in any of the previous embodiments, the primary, secondary, tertiary, or quaternary probe comprises at least two readout probe binding sites. In some embodiments, in any of the previous embodiments, the primary, secondary, tertiary, or quaternary probe comprises at least three readout probe binding sites. In some embodiments, in any of the previous embodiments, the primary, secondary, tertiary, or quaternary probe comprises at least four readout probe binding sites. In some embodiments, in any of the previous embodiments, the primary, secondary, tertiary, or quaternary probe comprises at least five readout probe binding sites. In some embodiments, in any of the previous embodiments, the primary, secondary, tertiary, or quaternary probe comprises at least six readout probe binding sites. In some embodiments, in any of the previous embodiments, the primary, secondary, tertiary, or quaternary probe comprises at least seven readout probe binding sites. In some embodiments, in any of the previous embodiments, the primary, secondary, tertiary, or quaternary probe comprises at least eight readout probe binding sites. In some embodiments, in any of the previous embodiments, the primary, secondary, tertiary, or quaternary probe comprises at least nine readout probe binding sites. In some embodiments, in any of the previous embodiments, the primary, secondary, tertiary, or quaternary probe comprises at least 10 readout probe binding sites.

In some embodiments, the methods of any of the previous embodiments comprises contacting a sample with two or more primary probes that bind one or more targets, wherein the two or more primary probes hybridize to the target.

In some embodiments, the methods of any of the preceding embodiments comprises, hybridizing a secondary probe to the ligated primary probe, wherein the secondary probe comprises two or more tertiary probe binding sites or two or more readout probe binding sites.

In some embodiments, the methods of any of the preceding embodiments comprises hybridizing a tertiary probe to at least two secondary probes; and wherein each tertiary probe comprises two or more readout probe binding sites.

In some embodiments, the methods of any of the preceding embodiments comprise hybridizing readout probes capable of detection to two or more readout-probe binding sites.

In some embodiments, the one or more primary probe in any of the previous embodiments comprises a padlock probe. In some embodiments, the one or more secondary probe in any of the previous embodiments comprises a padlock probe. In some embodiments, the one or more tertiary probe in any of the previous embodiments comprises a padlock probe. In some embodiments, the one or more quaternary probe in any of the previous embodiments comprises a padlock probe.

In some embodiments, the methods of any of the preceding embodiments comprise primary, secondary, tertiary, or quaternary probes having nucleotides at the 5′ and 3′ end that are complementary to their respective target sequences.

In some embodiments, the probes of any of the previous embodiments comprise oligonucleotides, oligonucleotide conjugated antibodies, oligonucleotides conjugated with other affinity reagents, or any combination thereof. The targets can be nucleic acids or protein or other cellular components. In some embodiments, the probes of any of the previous embodiments, interact with their target through protein-protein interactions, RNA-protein, RNA-RNA, RNA-DNA, DNA-protein, or any combination thereof.

### Stabilizing the Probes

In some embodiments, the method of any of the preceding embodiments comprise stabilizing the primary, secondary, tertiary probes, or quaternary probes. In some embodiments, the method comprises stabilizing the primary probe. In some embodiments, the method comprises stabilizing the secondary probe. In some embodiments, the method comprises stabilizing the tertiary probe. In some embodiments, the method further comprises stabilizing the quaternary probe.

In some embodiments, the probes are stabilized by methods selected from enzyme ligation, chemical ligation, UV crosslinking with or without oligo splint probes, hybridization of splint probes, crosslinking through a matrix, and chemical crosslinking, or any combination thereof. In certain embodiments, the enzymes used for enzyme ligation are selected from T4 Ligase, T7 Ligase, quick ligase, and ampligase. In certain embodiments, the enzyme is a DNA ligase. In certain embodiments, the enzyme is an RNA ligase. In certain embodiments, the chemical ligation is selected from comprise amine-phosphate, diamine, and thiol ligation. In certain embodiments, the crosslinking through the matrix comprises a hydrogel made from polyacrylamide or agarose. In certain embodiments, the chemical crosslinking for stabilization is selected from paraformaldehyde, glutaraldehyde, and reversible crosslinkers such as DSP (dithiobis succinimidyl propionate). In certain embodiments, the splint probes comprise locked nucleic acid (LNA) or peptide nucleic acid (PNA).

### Ligation

In some embodiments, the primary, secondary, or tertiary probes of any of the preceding embodiments are ligated or cross-linked 5′ to 3′ to form closed circles. In some embodiments, the primary, secondary, or tertiary probes of any of the preceding embodiments are cis-ligated 5′ to 3′ to form a closed circle.

In some embodiments, the methods of any of the preceding embodiments comprises a primary probe that is ligated or cross-linked either cis or trans 5′ to 3′ to form closed circles. In some embodiments, the methods of any of the preceding embodiments use a primary probe that is ligated or cross-linked 5′ to 3′ to form a closed circle.

In some embodiments, the primary, secondary, tertiary, or quaternary probes of any of the previous embodiments are ligated 5′ to 3′ by using a ligase.

In certain embodiments, the probes are ligated either cis or trans. In some embodiments, the primary, secondary, tertiary, or quaternary probes of any of the previous embodiments are cis-ligated 5′ to 3′. In some embodiments, the primary, secondary, tertiary, or quaternary probes of any of the previous embodiments are trans-ligated 5′ to 3′.

In some embodiments, the primary, secondary, tertiary, or quaternary probes of any of the previous embodiments are cis-ligated through one or more additional molecules, such as an oligonucleotide probe, LNA or PNA, or a protein, molecular complexes, or small chemical molecules.

In some embodiments, the primary, secondary, tertiary, or quaternary probes of any of the previous embodiments are UV cis-ligated either directly or indirectly through intermediate molecules.

In some embodiments, the primary, secondary, tertiary, or quaternary probes of any of the previous embodiments are cis-ligated with the cellular or sample matrix directly or through intermediate molecules. In certain embodiments, the primary, secondary, tertiary, or quaternary probes of any of the previous embodiments are cis-ligated using chemical crosslinkers comprising paraformaldehyde, glutaraldehyde, or reversible crosslinkers. In certain embodiments, the primary, secondary, tertiary, or quaternary probes of any of the previous embodiments are cis-ligated using a matrix comprising a native tissue matrix, tissue matrix, or an exogenous matrix. In certain embodiments the exogenous matrix comprises a hydrogel.

In some embodiments, the primary, secondary, tertiary, or quaternary probes of any of the previous embodiments are stabilized before a subsequent round of probe hybridization. In certain embodiments, the primary, secondary, tertiary, or quaternary probes of any of the previous embodiments are stabilized before a stripping step.

In some embodiments, the primary, secondary, tertiary, or quaternary probes of any of the previous embodiments are cis-ligated 5′ to 3′ by acrylamide polymerization.

In some embodiments, the primary, secondary, tertiary, or quaternary probes of any of the previous embodiments are cis-ligated 5′ to 3′ by click chemistry.

In some embodiments, the primary, secondary, tertiary, or quaternary probes of any of the previous embodiments are cis-ligated 5′ to 3′ by reactive groups on the 5′ and/or 3′ wherein the reactive groups form a reactive pair selected from alkenes, alkynes, azides, amides, amine, nitrones, phosphates, tetrazines, and tetrazoles.

### Readout Probes

In some embodiments, the compositions, kit, and methods comprise readout probes. In some embodiments, the one or more readout probes of any of the preceding embodiments comprise oligonucleotides with the same sequence.

In some embodiments, the one or more readout probes of any of the preceding embodiments comprise oligonucleotides with different sequences.

In some embodiments, the one or more readout probes of any of the preceding embodiments comprise oligonucleotides that are at least 17 nucleotides in length.

In some embodiments, the readout probe of any of the preceding embodiments comprises oligonucleotides that are at least 5 nucleotides in length. In some embodiments, the readout probe of any of the preceding embodiments comprises oligonucleotides that are at least 10 nucleotides in length In some embodiments, the readout probe of any of the preceding embodiments comprises oligonucleotides that are at least 11 nucleotides in length. In some embodiments, the readout probe of any of the preceding embodiments comprises oligonucleotides that are at least 12 nucleotides in length. In some embodiments, the readout probe of any of the preceding embodiments comprises oligonucleotides that are at least 13 nucleotides in length. In some embodiments, the readout probe of any of the preceding embodiments comprises oligonucleotides that are at least 14 nucleotides in length. In some embodiments, the readout probe of any of the preceding embodiments comprises oligonucleotides that are at least 15 nucleotides in length. In some embodiments, the readout probe of any of the preceding embodiments comprises oligonucleotides that are at least 16 nucleotides in length. In some embodiments, the readout probe of any of the preceding embodiments comprises oligonucleotides that are at least 17 nucleotides in length. In some embodiments, the readout probe of any of the preceding embodiments comprises oligonucleotides that are at least 18 nucleotides in length. In some embodiments, the readout probe of any of the preceding embodiments comprises oligonucleotides that are at least 19 nucleotides in length. In some embodiments, the readout probe of any of the preceding embodiments comprises oligonucleotides that are at least 20 nucleotides in length. In some embodiments, the readout probe of any of the preceding embodiments comprises oligonucleotides that are at least 21 nucleotides in length. In some embodiments, the readout probe of any of the preceding embodiments comprises oligonucleotides that are less than 30, 50, 100, 200, 250, 500, 750, or 1000 nucleotides in length.

In some embodiments, the secondary probe complements the secondary probe binding site on the primary probe. In some embodiments, the tertiary probe complements the secondary probe binding site on the secondary probe. In some embodiments, the quarternary probe complements the tertiary probe binding site on the tertiary probe. In some embodiments, the probe complements comprise a sequence complementarity that is at least 60%, 65%, 70%, 75%, 80%, 85%, 90%, 91%, 92%, 93%, 94%, 95%, 96%, 97%, 98%, 99%, or 100%.

In some embodiments, the length of the primary, secondary, tertiary, and quaternary probe binding sites range from 5-100 nucleotides. In some embodiments, the length of the primary, secondary, tertiary, and quaternary probe binding sites range from 5-10 nucleotides. In some embodiments, the length of the primary, secondary, tertiary, and quaternary probe binding sites range from 5-20 nucleotides. In some embodiments, the length of the primary, secondary, tertiary, and quaternary probe binding sites range from 5-30 nucleotides. In some embodiments, the length of the primary, secondary, tertiary, and quaternary probe binding sites range from 5-40 nucleotides. In some embodiments, the length of the primary, secondary, tertiary, and quaternary probe binding sites range from 5-50 nucleotides. In some embodiments, the length of the primary, secondary, tertiary, and quaternary probe binding sites range from 5-60 nucleotides. In some embodiments, the length of the primary, secondary, tertiary, and quaternary probe binding sites range from 5-70 nucleotides. In some embodiments, the length of the primary, secondary, tertiary, and quaternary probe binding sites range from 5-80 nucleotides. In some embodiments, the length of the primary, secondary, tertiary, and quaternary probe binding sites range from 5-90 nucleotides.

In some embodiments, the readout probe complements the readout probe binding site on the primary probe. In some embodiments, the readout probe complements the readout probe binding site on the secondary probe. In some embodiments, the readout probe complements the readout probe binding site on the tertiary probe. In some embodiments, the readout probe complements the readout probe binding site on the quaternary probe. In some embodiments, the probe complements comprise a sequence complementarity that is at least 60%, 65%, 70%, 75%, 80%, 85%, 90%, 91%, 92%, 93%, 94%, 95%, 96%, 97%, 98%, 99%, or 100%.

In some embodiments, the length of the readout probe binding sites range from 5-100 nucleotides. In some embodiments, the length of the readout probe binding sites range from 5-10 nucleotides. In some embodiments, the length of the readout probe binding sites range from 5-20 nucleotides. In some embodiments, the length of the readout probe binding sites range from 5-30 nucleotides. In some embodiments, the length of the readout probe binding sites range from 5-40 nucleotides. In some embodiments, the length of the readout probe binding sites range from 5-50 nucleotides. In some embodiments, the length of the readout probe binding sites range from 5-60 nucleotides. In some embodiments, the length of the readout probe binding sites range from 5-70 nucleotides. In some embodiments, the length of the readout probe binding sites range from 5-80 nucleotides. In some embodiments, the length of the readout probe binding sites range from 5-90 nucleotides.

In some embodiments, the methods of any of preceding embodiments, further comprises repeating the contacting and imaging steps, each time with a new plurality of detectably labeled readout probes, wherein in each new plurality at least one readout probe for one target differs from at least one readout probe for the same target in a previous plurality, wherein they differ at least in their detectable moieties.

In some embodiments, the one or more readout probes of any of the previous embodiments comprise an oligonucleotide or antibody with a detectable moiety. In some embodiments, the one or more readout probes of any of the previous embodiments comprises an oligonucleotide, protein, antibody, or any combination thereof.

In some embodiments, the methods of any of preceding embodiments, further comprises repeating the contacting and imaging steps, each time with a new plurality of detectably labeled readout probes, wherein in each new plurality at least one readout probe for one target differs from at least one readout probe for the same target in a previous plurality, wherein they differ at least in their detectable moieties.

### Detectable Moiety

In some embodiments, the detectable moiety is any fluorophore deemed suitable by those of skill in the arts. In some embodiments, the readout probe comprises one or more detectable moieties. In certain embodiments, the detectable moieties are the same. In certain embodiments, the detectable moieties are different. In some embodiments, the methods of any one of the preceding embodiments comprises at least two different detectable moieties.

In some embodiments, the detectable moieties include but are not limited to fluorescein, rhodamine, Alexa Fluors, DyLight fluors, ATTO Dyes, or any analogs or derivatives thereof. In certain embodiments, the detectable moieties include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red).

In some embodiments, the detectable moieties include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl) naphthacene; 9,10-Bis(phenylethynyl)anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl)anthracene; 2-Chloro-9,10-bis(phenylethynyl)anthracene; 2-Chloro-9,10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris (bathophenanthroline disulfonate) ruthenium (II), Texas Red, TSQ, Umbelliferone, or Yellow fluorescent protein.

In some embodiments, the detectable moieties include but are not limited to Alexa Fluor family of fluorescent dyes (Molecular Probes, Oregon). Alexa Fluor dyes are widely used as cell and tissue labels in fluorescence microscopy and cell biology. The excitation and emission spectra of the Alexa Fluor series cover the visible spectrum and extend into the infrared. The individual members of the family are numbered according roughly to their excitation maxima (in nm). Certain Alexa Fluor dyes are synthesized through sulfonation of coumarin, rhodamine, xanthene (such as fluorescein), and cyanine dyes. In some embodiments, sulfonation makes Alexa Fluor dyes negatively charged and hydrophilic. In some embodiments, Alexa Fluor dyes are more stable, brighter, and less pH-sensitive than common dyes (e.g. fluorescein, rhodamine) of comparable excitation and emission, and to some extent the newer cyanine series. Exemplary Alexa Fluor dyes include but are not limited to Alexa-350, Alexa-405, Alexa-430, Alexa-488, Alexa-500, Alexa-514, Alexa-532, Alexa-546, Alexa-555, Alexa-568, Alexa-594, Alexa-610, Alexa-633, Alexa-647, Alexa-660, Alexa-680, Alexa-700, or Alexa-750.

In some embodiments, the detectable moieties comprise one or more of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, the detectable moieties comprises a nanomaterial. In some embodiments, the fluorophore is a nanoparticle. In some embodiments, the detectable moiety is or comprises a quantum dot. In some embodiments, the fluorophore is a quantum dot. In some embodiments, the detectable moiety comprises a quantum dot. In some embodiments, the detectable moiety is or comprises a gold nanoparticle. In some embodiments, the detectable moiety is a gold nanoparticle. In some embodiments, the detectable moiety comprises a gold nanoparticle.

### Composition and Kit

In some embodiments, the composition or kit comprises at least two different detectable moieties.

In some embodiments, the composition or kit of any of the preceding embodiments comprises two or more primary probes capable of binding two or more targets.

In some embodiments, the composition or kit of any of the preceding embodiments comprises two or more secondary probes capable of binding the primary probe, wherein each secondary probe comprises two or more tertiary probe binding sites or two or more readout probe binding sites.

In some embodiments, the composition or kit of any of the preceding embodiments comprises two or more tertiary probes, each capable of binding to two or more tertiary probe binding sites, and wherein each tertiary probe comprises one or more readout probe binding sites.

In some embodiments, the composition or kit of any of the previous embodiments comprises two or more readout probes capable of binding to at least one of the one or more readout probe binding site and capable of being detected.

In some embodiments, the kit of any of the previous embodiments comprises a DNA ligase. In certain embodiments, the kit of any of the previous embodiments, comprises an RNA ligase.

### Washes and Removing Probes

In some embodiments, the method of any of the preceding embodiments, comprises washing the sample after each step. In certain embodiments, the sample is washed with a buffer that removes non-specific hybridization reactions. In certain embodiments, formamide is used in the wash step. In certain embodiments, the wash buffer is stringent. In certain embodiments, the wash buffer comprises 10% formamide, 2×SSC, and 0.1% triton X-100s.

In some embodiments, the methods of any of the previous embodiments comprise a step of removing a plurality of detectably labeled oligonucleotides after each imaging step. In some embodiments, the methods of any of the previous embodiments comprise a step of removing comprises contacting the plurality of detectably labeled oligonucleotides with a DNase, contacting the plurality of detectably labeled oligonucleotides with an RNase, photobleaching, strand displacement, formamide wash, or any combinations thereof.

### Imaging the Probes

In some embodiments, the method comprises detecting the probes by imaging the probes. As understood by a person having ordinary skill in the art, different technologies can be used for the imaging steps.

In some embodiments, the imaging methods comprise but are not limited to epi-fluorescence microscopy, confocal microscopy, the different types of super-resolution microscopy (PALM/STORM, SSIM/GSD/STED), and light sheet microscopy (SPIM and etc).

In some embodiments, the imaging methods comprise exemplary super resolution technologies include, but are not limited to I⁵M and 4Pi-microscopy, Stimulated Emission Depletion microscopy (STEDM), Ground State Depletion microscopy (GSDM), Spatially Structured Illumination microscopy (SSIM), Photo-Activated Localization Microscopy (PALM), Reversible Saturable Optically Linear Fluorescent Transition (RESOLFT), Total Internal Reflection Fluorescence Microscope (TIRFM), Fluorescence-PALM (FPALM), Stochastical Optical Reconstruction Microscopy (STORM), Fluorescence Imaging with One-Nanometer Accuracy (FIONA), and combinations thereof. For examples: Chi, 2009 “Super-resolution microscopy: breaking the limits,” Nature Methods 6(1): 15-18; Blow 2008, “New ways to see a smaller world,” Nature 456:825-828; Hell, et al, 2007, “Far-Field Optical Nanoscopy,” Science 316:1153; R. Heintzmann and G. Ficz, 2006, “Breaking the resolution limit in light microscopy,” Briefings in Functional Genomics and Proteomics 5(4):289-301; Garini et al., 2005, “From micro to nano: recent advances in high-resolution microscopy,” Current Opinion in Biotechnology 16:3-12; and Bewersdorf et al, 2006, “Comparison of I⁵M and 4Pi-microscopy,” 222(2): 105-1 17; and Wells, 2004, “Man the Nanoscopes,” JCB 164(3):337-340.

In some embodiments, electron microscopes (EM) are used for imaging.

In some embodiments, an imaging step detects a target. In some embodiments, an imaging step localizes a target. In some embodiments, an imaging step provides three-dimensional spatial information of a target. In some embodiments, an imaging step quantifies a target. By using multiple contacting and imaging steps, provided methods are capable of providing spatial and/or quantitative information for a large number of targets in surprisingly high throughput.

Certain techniques for imaging are known in the art. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

In some embodiments, the method comprises analyzing cell size and shape, markers, immunofluorescence measurements, or any combinations thereof.

### Exonucleases

In some embodiments, the methods of any of the preceding embodiments, comprise removing one or more probes bound non-specifically to targets, primary, secondary, tertiary, or quaternary probes with one or more exonucleases.

In some embodiments, the method of any of the preceding embodiments, comprises one or more exonucleases selected from the group consisting of Exonuclease I, Exonuclease III, Exonuclease T, Exonuclease V (RecBCD), Exonuclease VII, Exonuclease VIII, truncated, Lambda Exonuclease, Micrococcal, Nuclease, Mismatch Endonuclease I, Mung Bean Nuclease, Nuclease P1, RecJf, T5 Exonuclease, T7 Exonuclease, Thermolabile Exonuclease I.

The following non-limiting methods are provided to further illustrate the embodiments of the invention disclosed herein. It should be appreciated by those of skill in the art that the techniques disclosed in the examples that follow represent approaches that have been found to function well in the practice of several embodiments of the invention, and thus be considered to constitute examples of modes for its practice. However, those of skill in the art should, in light of the present disclosure, appreciate that many changes can be made in the specific embodiments that are disclosed and still obtain a like or similar result without departing from the spirit and the scope of the invention.

### METHODS

### Experiment 1

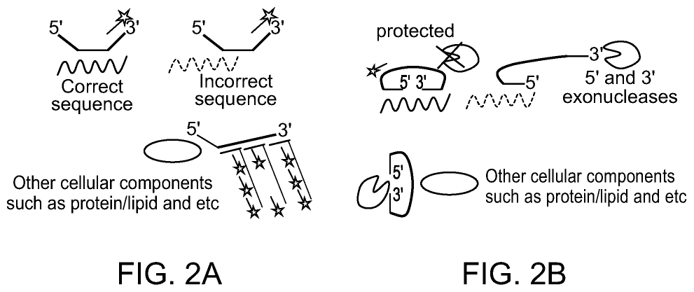

Conventional primary probes in FISH experiments (FIG. 1a) as well as amplification methods such as in branched DNA, SABER, are prone to non-specific binding on the wrong target sequences, such as partial binding to RNA in cell culture or tissue samples, because any non-specific binding can be readout by the fluorescent readout probes (FIG. 2a). We reason that the usage of padlock probes as both primary probes and amplifiers together with 5′ and 3′ exonucleases can improve the signal to noise ratio of fluorescent signals (FIG. 1b). When the padlock probes/amplifiers bind correctly to the target sequences, both 5′ and 3′ end of the probes are protected from the exonucleases digestion, while partial hybridization of the probes/amplifiers to the wrong sequences, as well as non-specific between the probes/amplifiers with other cellular components, will be susceptible to these exonucleases digestion, removing the possibilities of the readout sites to be probed by the fluorescent readout (FIG. 2b). This non-specific binding removal strategy is key to signal amplification because otherwise nonspecific bound primary probes and amplifiers will be further amplified and overwhelm the specific signal. The hybridization of the amplifiers can be repeated as many rounds as desired to achieve more fold increase in fluorescent signals (FIG. 3).

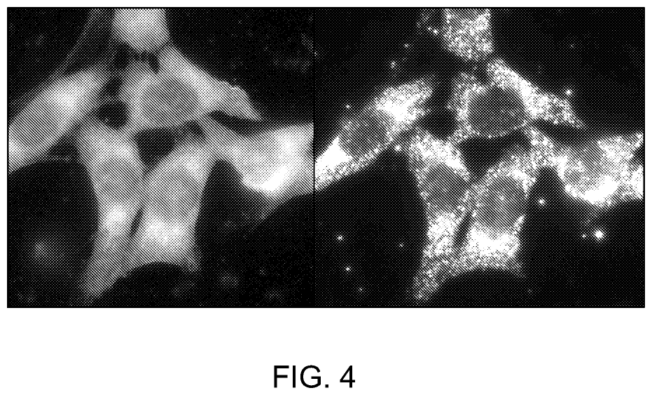

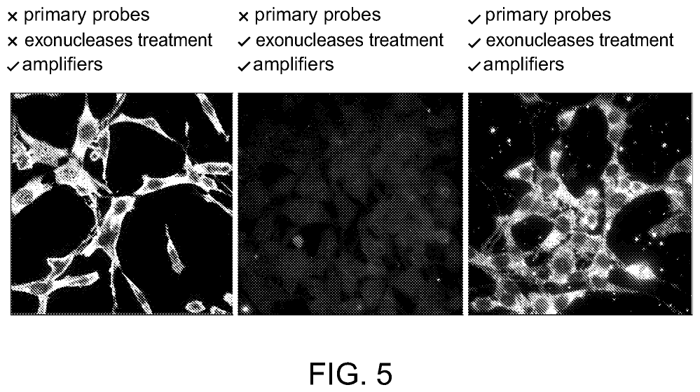

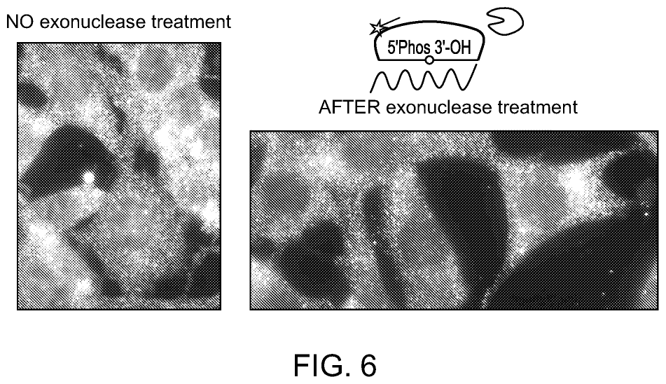

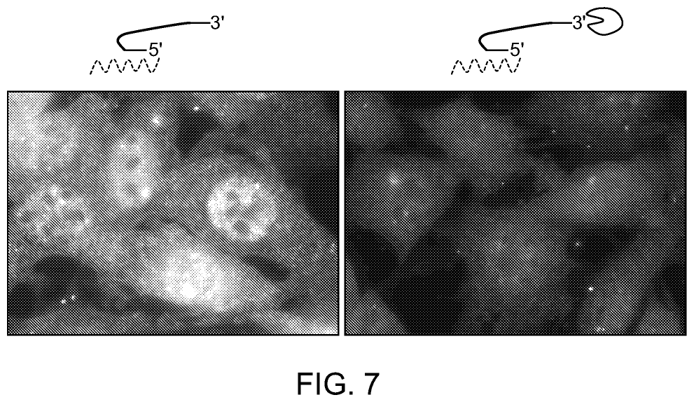

We first compare the fluorescent signals obtained from directly hybridizing to the primary probes vs after 6 rounds of amplification (FIG. 4). We showed that by performing multiple rounds of hybridization of amplifiers, followed by DNA-specific exonuclease treatment, improves the signal-to-noise ratio of the fluorescent dots dramatically. The images are shown as the same contrast in FIG. 4. To show the exonuclease treatment decreases the non-specific staining of the amplifiers, we used one of the amplifiers that was found to stick non-specifically to cellular background and caused high fluorescent signals despite primary probes not being hybridized to the target as control. Surprisingly, by incorporating exonuclease treatment, these nonspecific backgrounds are reduced close to cellular background when primary probes are not hybridized (FIG. 5). When primary probes are hybridized, amplified, and exonuclease treated, the same amplifier now contributes to the detection of true positive signals. Next, to show primary padlock probes on RNA are also protected when perfectly hybridized to the target sequence, we design padlock probes with perfect and imperfect binding sites on one side of the probes. The perfect padlock probes will hybridize 15 nucleotide+15 nucleotide perfectly to the target gene Eef2, while the imperfect padlock probes will hybridize with 15 nucleotides+15 random nucleotides. FIG. 6 shows that perfectly hybridized primary padlock probes are protected from DNA-specific exonuclease treatment. FIG. 7 shows the imperfect padlock probes has non-specific nuclear staining and in cytoplasm, and the usage of DNA-specific exonucleases remove those non-specific bindings. In the fixed cell with high complexity components, it is surprising that exonucleases are highly specific and processive in degrading non-specific bound probes while leaving RNA and specifically bound probes intact.

### Example 2

Oligonucleotides targeting different targets (spliceosome RNA and an mRNA) were used to detect the interaction between the spliceosome RNA and the mRNA.

Once bound to the spliceosome RNA and mRNA, the oligonucleotides were ligated to each other, preventing degradation by exonuclease.

The ligated probes were amplified by RCA or LANTERN allowing the signal from targets that are interacting or proximal to be detected.

### REFERENCES

The following references are incorporated by their entirety.
Agouridas, V., El Mahdi, O., Diemer, V., Cargoët, M., Monbaliu, J. M., & Melnyk, O. (2019). Native Chemical Ligation and Extended Methods: Mechanisms, Catalysis, Scope, and Limitations. Chemical Reviews, 119(12).
Bauer, R. J., Zhelkovsky, A., Bilotti, K., Crowell, L. E., Evans, T. C., Jr, McReynolds, L. A., & Lohman, G. J. S. (2017). Comparative analysis of the end-joining activity of several DNA ligases. PloS One, 12(12), e0190062.
Bharti, A., Rashmi, T., Pankaj, A., & Rohit, B. (2017). Chemical Crosslinking: Role in Protein and Peptide Science. Current Protein & Peptide Science, 18 (9), 946-955.
Bi, X., & Liang, A. (2016). Emerging Concepts in Analysis and Applications of Hydrogels. In Emerging Concepts in Analysis and Applications of Hydrogels. IntechOpen.
Braasch, D. A., & Corey, D. R. (2001). Locked nucleic acid (LNA): fine-tuning the recognition of DNA and RNA. Chemistry & Biology, 8(1).
Chen, F., Tillberg, P. W., & Boyden, E. S. (2015). Expansion microscopy. Science, 347(6221), 543-548.
Chen, F., Wassie, A. T., Cote, A. J., Sinha, A., Alon, S., Asano, S., Daugharthy, E. R., Chang, J. B., Marblestone, A., Church, G. M., Raj, A., & Boyden, E. S. (2016). Nanoscale imaging of RNA with expansion microscopy. Nature Methods, 13(8).
Chen, H., Du, F., Chen, G., Streckenbach, F., Yasmeen, A., Zhao, Y., & Tang, Z. (2014). Template-directed Chemical Ligation to Obtain 3′-3′ and 5′-5′ Phosphodiester DNA Linkages. Scientific Reports, 4(1), 1-6. Cross-link. (n.d.).
El-Sagheer, A. H., & Brown, T. (2017). Single tube gene synthesis by phosphoramidate chemical ligation. Chemical Communications, 53(77), 10700-10702.
Eng, C.-H. L., Lawson, M., Zhu, Q., Dries, R., Koulena, N., Takei, Y., Yun, J., Cronin, C., Karp, C., Yuan, G.-C., & Cai, L. (2019). Transcriptome-scale super-resolved imaging in tissues by RNA seqFISH. Nature, 568(7751), 235-239.
Eng, C.-H. L., Shah, S., Thomassie, J., & Cai, L. (2017). Profiling the transcriptome with RNA SPOTs. Nature Methods, 14(12), 1153-1155.
Ghesquière, J., Gauthier, N., De Winter, J., Gerbaux, P., Moucheron, C., Defrancq, E., & Kirsch-De, M. A. (2012). Photocrosslinking between peptide-peptide or peptide-oligonucleotide by Ru (II)-TAP complexes. Chemistry, 18(1).
Haque, M. M., & Peng, X. (2013). DNA-associated click chemistry. Science China. Chemistry, 57(2), 215-231.
Jin, J., Vaud, S., Zhelkovsky, A. M., Posfai, J., & McReynolds, L. A. (2016). Sensitive and specific miRNA detection method using SplintR Ligase. Nucleic Acids Research, 44(13).
Lubeck, E., Coskun, A. F., Zhiyentayev, T., Ahmad, M., & Cai, L. (2014). Single-cell in situ RNA profiling by sequential hybridization. Nature Methods, 11(4), 360-361.
Nichols, N. M., Tabor, S., & McReynolds, L. A. (2008). RNA ligases. Current Protocols in Molecular Biology/Edited by Frederick M. Ausubel . . . [et Al.], Chapter 3.
Nowak-Karnowska, J., Zielińska, K., Milecki, J., & Skalski, B. (2021). Thermally reversible and irreversible interstrand photocrosslinking of 5-chloro-2′-deoxy-4-thiouridine modified DNA oligonucleotides. Organic & Biomolecular Chemistry, 19(6), 1292-1295.
Pellestor, F., & Paulasova, P. (2004). The peptide nucleic acids (PNAs), powerful tools for molecular genetics and cytogenetics. European Journal of Human Genetics: EJHG, 12(9), 694-700.
Shah, S., Lubeck, E., Schwarzkopf, M., He, T.-F., Greenbaum, A., Sohn, C. H., Lignell, A., Choi, H. M. T., Gradinaru, V., Pierce, N. A., & Cai, L. (2016). Single-molecule RNA detection at depth by hybridization chain reaction and tissue hydrogel embedding and clearing. Development, 143(15), 2862.
Shah, S., Lubeck, E., Zhou, W., and Cai, L. (2016). In Situ Transcription Profiling of Single Cells Reveals Spatial Organization of Cells in the Mouse Hippocampus. Neuron, 92(2), 342-357.
Shah, S., Takei, Y., Zhou, W., Lubeck, E., Yun, J., Eng, C.-H. L., Koulena, N., Cronin, C., Karp, C., Liaw, E. J., Amin, M., & Cai, L. (2018). Dynamics and Spatial Genomics of the Nascent Transcriptome by Intron seqFISH. Cell, 174(2), 363-376.e16.
Shuman, S. (2009). DNA Ligases: Progress and Prospects. The Journal of Biological Chemistry, 284(26), 17365.
Tillberg, P. W., Chen, F., Piatkevich, K. D., Zhao, Y., Yu, C.-C. (jay), English, B. P., Gao, L., Martorell, A., Suk, H.-J., Yoshida, F., DeGennaro, E. M., Roossien, D. H., Gong, G., Seneviratne, U., Tannenbaum, S. R., Desimone, R., Cai, D., & Boyden, E. S. (2016). Protein-retention expansion microscopy of cells and tissues labeled using standard fluorescent proteins and antibodies. Nature Biotechnology, 34(9), 987-992.
Yang, B., Treweek, J. B., Kulkarni, R. P., Deverman, B. E., Chen, C.-K., Lubeck, E., Shah, S., Cai, L., & Gradinaru, V. (2014). Single-Cell Phenotyping within Transparent Intact Tissue Through Whole-Body Clearing. Cell, 158(4), 945.

1. A composition to reduce the background signal of probes hybridized to targets, wherein the composition comprises:
(i) one or more primary probes capable of binding one or more targets, wherein each primary probe comprises one or more secondary probe binding sites and/or one or more readout probe binding sites;
(ii) optionally, one or more secondary probes, each capable of binding the primary probe or one or more targets, wherein each secondary probe comprises one or more tertiary probe binding sites or one or more readout probe binding sites;
(iii) optionally, one or more tertiary probes, each capable of binding to the secondary probe or one or more targets, wherein each tertiary probe comprises one or more quaternary probe binding sites or one or more readout probe binding sites;
(iv) optionally, one or more quaternary probes, each capable of binding to the tertiary probe or one or more targets, wherein each quaternary probe comprises one or more readout probe binding sites; and
(v) one or more readout probes capable of binding to a readout probe binding site on the one or more primary, secondary, tertiary, or quaternary probes and capable of being detected.
(vi) one or more exonucleases each capable of removing one or more probes that bind to targets, primary, secondary, tertiary, or quaternary probes.

2. (canceled)

3. A method to reduce the background signal of probes hybridized to targets, comprising steps of:
(i) contacting a sample with one or more primary probes that bind one or more targets, wherein each primary probe hybridizes to a target, wherein each primary probe comprises one or more secondary probe binding sites and/or one or more readout probe binding sites;
(ii) optionally, hybridizing one or more secondary probes to the primary probes or to one or more targets; wherein each secondary probe comprises one or more tertiary probe binding sites or one or more readout probe binding sites;
(iii) optionally, hybridizing one or more tertiary probes to at least one secondary probe or one or more targets, wherein each tertiary probe comprises one or more quaternary probe binding sites or one or more readout probe binding sites;
(iv) optionally, hybridizing one or more quaternary probes to at least one tertiary probe or one or more targets, wherein each quaternary probe comprises one or more readout probe binding sites;
(v) hybridizing readout probes capable of detection to the one or more readout probe binding sites;
(vi) contacting the sample one or more exonucleases each capable of removing probes that bind to targets, primary, secondary, tertiary, or quaternary probes after any steps (i)-(v) to reduce the background signal of probes bound to targets.

4. The method of claim 3, wherein any of the steps (i)-(vii) are repeated either individually or in any combination thereof.

5. The method of claim 3, further comprising stabilizing the primary, secondary, tertiary, or quaternary probes.

6. The method of claim 3, further comprising stabilizing the primary probe.

7. The method of claim 3, further comprising stabilizing the secondary probe.

8. The method of claim 3, further comprising stabilizing the tertiary probe.

9. The method of claim 3, further comprising stabilizing the quaternary probe.

10. The method of claim 5, wherein the stabilizing is selected from the group consisting of enzyme ligation, chemical ligation, UV crosslinking with or without oligo splint probes, hybridization of splint probes, crosslinking through a matrix, and chemical crosslinking, and any combination thereof.

11. The method of claim 3 wherein the targets are selected from transcripts, RNA, DNA loci, chromosomes, DNA, proteins, lipids, glycans, cellular targets, organelles, and any combinations thereof.

12. The targets of claim 11, wherein the targets are conjugated to one or more oligonucleotide sequences.

13. (canceled)

14. The method of claim 3, wherein the primary, secondary, tertiary, or quaternary probes are cis-ligated 5′ to 3′ with an enzyme.

15.-18. (canceled)

19. The method of claim 3 wherein the primary, secondary, tertiary, or quaternary probes are cis-ligated 5′ to 3′ by click chemistry.

20. The method of claim 3 wherein the primary, secondary, tertiary, or quaternary probes are cis-ligated 5′ to 3′ by reactive groups on the 5′ and/or 3′ ends wherein the reactive groups form a reactive pair selected from alkenes, alkynes, azides, amides, amines, nitrones, and phosphates, tetrazines, and tetrazoles.

21. The method of claim 3, wherein one or more of the readout probes comprise an oligonucleotide or antibody with a detectable moiety.

22.-35. (canceled)

36. The method of claim 3 further comprising:
(vii) imaging the cell after step (vi) so that the interaction of the primary probe to the nucleic acids is detected; and
(viii) optionally repeating the contacting and imaging steps, each time with a new plurality of detectably labeled readout probes, so that a target nucleic acid in the sample/cell is described by a barcode, determined by the repeated contacting and imaging steps, that can be differentiated from the barcodes of the other target nucleic acids in the cell.

37. The method of claim 36, comprising repeating the contacting and imaging steps, each time with a new plurality of detectably labeled readout probes.

38. The method of claim 37, wherein in each new plurality at least one readout probe for one target differs from at least one readout probe for the same target in a previous plurality.

39. The method of claim 37, wherein each readout probe in the plurality of detectably labeled readout probes differs in at least in their detectable moieties.

40. (canceled)

41. The method of claim 3, wherein the primary, secondary, or tertiary probes are ligated or cross-linked, either cis or trans, 5′ to 3′ to form a closed circle.

42. (canceled)

43. The method of claim 3, wherein the one or more exonucleases is selected from the group consisting of Exonuclease I, Exonuclease III, Exonuclease T, Exonuclease V (RecBCD), Exonuclease VII, Exonuclease VIII, truncated, Lambda Exonuclease, Micrococcal, Nuclease, Mismatch Endonuclease I, Mung Bean Nuclease, Nuclease P1, RecJf, T5 Exonuclease, T7 Exonuclease, Thermolabile Exonuclease I.

44. The method of claim 36, further comprising a step of removing a plurality of detectably labeled oligonucleotides after each imaging step.

45. The method of claim 44, wherein the method further comprises a step of removing comprises contacting the plurality of detectably labeled oligonucleotides with a DNase, contacting the plurality of detectably labeled oligonucleotides with an RNase, photobleaching, strand displacement, formamide wash, or combinations thereof.

46.-58. (canceled)

59. The method of claim 3, wherein the exonucleases remove one or more probes bound non-specifically to targets, primary, secondary, tertiary, or quaternary probes.

60. The method of claim 3, further comprises:
(vii) ligating probes in close proximity before step (v);
(viii) treating the ligated probes with an exonuclease;
(ix) optionally, enhancing the signals of probes that are not cleaved after step (viii) by LANTERN, RCA, branched DNA assay, HCR, SABER, STARMAP, or other in situ amplification methods; and
(x) determining the proximity of the probes interacting with their targets to each other.

61. The method of claim 60, wherein the interaction comprises glycans, cellular components, protein-protein interactions, RNA-protein interactions, RNA-RNA, RNA-DNA, DNA-protein, or combinations thereof.
